(19) United States
(12) Patent Application Publication
(10) Pub. No.: US 2021/0157459 A1
(43) Pub. Date: May 27, 2021

(54) METHODS AND SYSTEMS FOR DISPLAYING ASSOCIATIONS AND TIMELINES OF MEDICAL STUDIES

(71) Applicant: Fujifilm Medical Systems U.S.A., Inc., Morrisville, NC (US)

(72) Inventor: Jeffrey Cohee, Noblesville, IN (US)

(21) Appl. No.: 16/697,866

(22) Filed: Nov. 27, 2019

(51) Int. Cl. G06F 3/0482; G16H 30/20; G16H 30/40; G06N 20/00; G06F 3/0481; G06F 3/0485

(52) U.S. Cl. CPC G06F 3/0482; G16H 30/20; G06F 3/0485; G06N 20/00; G06F 3/04817; G16H 30/40

(57) ABSTRACT

A method for displaying a list of studies associated with a patient, including, receiving a request to view two or more studies associated with the patient; displaying, on a graphical user interface, a list of the studies associated with the patient, the list including a study icon associated with each study; receiving a user selection of a first study from the studies associated with the patient; modifying, on the graphical user interface, the appearance of a first study icon associated with the first study displaying, on the graphical user interface, at least one image associated with the first study; identifying one or more related studies from the studies associated with the patient, each related study being associated with the first study; and modifying, on the graphical user interface, the appearance of at least one study icon associated with one of the one or more related studies.

### BACKGROUND

### 1. Field of Disclosed Subject Matter

he disclosed subject matter is directed to methods and systems for display of medical images and information relevant to one or more of the displayed images. More specifically, the methods and systems can identify and display associations between multiple medical studies associated with a particular patient and the methods and systems can create and display timelines of medical studies associated with a particular patient.

### 2. Description of Related Art

Medical imaging technology includes the use of medical images such as, among others, x-rays (or x-rays' digital counterparts: computed radiography (CR) and digital radiography (DR)), mammograms, computerized tomography (CT) scans, magnetic resonance images (MRIs), positron emission tomography (PET) scans and ultrasound images. Some medical facilities, such as doctors' offices, dentists' offices, hospitals, etc., can use x-ray illuminators to view physical printouts of medical images. However, medical facilities are adopting electronic displays for displaying medical images.

There are several advantages to switching to electronic displays. One exemplary advantage of switching to electronic displays is that adjusting viewing properties for one or more medical images has become easier (e.g., attributes of the one or more medical images). For example, adjusting the brightness level of an image on a computer for display on the display screen is easier and more convenient than adjusting the light source of an x-ray illuminator. More information on adjusting viewing properties of electronic images can be found in U.S. Pat. No. 10,417,326 and U.S. Application Publication No. 2017/0039320, each of which is incorporated herein by reference.

However, as medical facilities adopt electronic displays, medical personnel, such as doctors, nurses, or medical technicians, can have difficulty accessing a plurality of pieces of medical information at once. Previously, with x-ray illuminators, for example, a plurality of physical x-ray image films could be hung against a backlit screen of an x-ray illuminator. Additionally, medical personal had the ability to hang other relevant medical information, such as medical records, charts, surgery procedures, etc., in a side-by-side manner with one or more x-rays.

Electronic displays to display medical information can require medical personnel to open a separate display screen for each piece of medical information. This can require the medical personnel reviewing or explaining the medical information to continually switch amongst the open windows, which can be clumsy and confusing for the medical personnel as well as for the viewer. For example, a doctor can be explaining an injury using a plurality of x-rays to a patient. By switching between the plurality of open windows, one for each x-ray, it is foreseeable that the doctor and/or patient will become confused, or the patient might not fully understand the injury and the potential treatment options. Furtheimore, it is possible for the medical personnel to select the incorrect image and create further confusion.

Accordingly, there is a need to provide an improved graphical user interface (GUI) for viewing electronic display of medical information. Particularly, there is a need to provide a user with a way to highlight and sort studies based on associations between the studies. Additionally, there is a need to allow a user to view patient level activity in a chronological format, while simultaneously being able to view individual study level activity of a patient. The user can then identify where a specific patient activity currently being reviewed falls within the patient's overall clinical history.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to systems and methods for displaying associations and timelines of medical studies. For example, a method for displaying a list of studies associated with a patient includes receiving, by one or more computing devices, a request to view two or more studies associated with the patient; displaying, by the one or more computing devices and on a GUI, a list of the studies associated with the patient, the list including a study icon associated with each study; receiving a user selection of a first study from the studies associated with the patient; modifying, by the one or more computing devices and on the GUI, the appearance of a first study icon associated with the first study; displaying, by the one or more computing devices and on the GUI, at least one image associated with the first study; identifying, by the one or more computing devices, one or more related studies from the studies associated with the patient, each related study being associated with the first study; and modifying, by the one or more computing devices and on the GUI, the appearance of at least one study icon associated with one of the one or more related studies.

In accordance with the disclosed subject matter, each study icon can display information regarding the respective associated study. The displayed information can include one or more of a date, a modality, an anatomical structure, a referring physician, and a thumbnail of the study. The method can include receiving a user request to change the displayed information and changing the displayed information based on the user request. Modifying the appearance of the study icon can include changing a color of the study icon.

Identifying one or more related studies from the studies associated with the patient can be based at least in part on a procedure code associated with each study. Identifying one or more related studies from the studies associated with the patient can be based at least in part on an anatomical structure associated with each study. Additionally or alternatively, identifying one or more related studies from the studies associated with the patient can be based at least in part on a manual association. Additionally or alternatively, identifying one or more related studies from the studies associated with the patient can be based at least in part on a machine learning model.

In accordance with the disclosed subject matter, a method for displaying a list of studies associated with a patient includes receiving, by one or more computing devices, a request to view the studies associated with the patient, each study being associated with a year; displaying, by the one or more computing devices and on a GUI, a scrollable list of the studies associated with the patient, the scrollable list including a study icon associated with each study; identifying, by the one or more computing devices, a beginning year, the beginning year being an earliest year associated with the studies; identifying, by the one or more computing devices, an ending year, the ending year being a latest year associated with the studies; and displaying, by the one or more computing devices and on the GUI, a non-scrollable timeline, the non-scrollable timeline including a year icon associated with each year including and between the beginning year and the ending year.

Each study icon can display information regarding the respective associated study. The information can include one or more of a date, a modality, an anatomical structure, a referring physician, and a thumbnail of the study.

The method can include receiving a user request to change the displayed information and changing the displayed information based on the user request. Each year icon associated with a year that is associated with at least one study can display a number-of-studies indicator. The number-of-studies indicator can be a bar having a length. The length can be based on a number of studies associated with the respective year. The bar lengths can be dynamically generated based on the number of studies associated with each year.

The method can include receiving a user selection of a first year icon associated with a selected year; and scrolling the scrollable list of studies to display study icons associated with the selected year at a top of the scrollable list.

The method can include receiving a user selection of a first study from the studies associated with the patient. The method can include modifying, by the one or more computing devices and on the GUI, the appearance of the year icon associated with the year associated with the first study.

In accordance with the disclosed subject matter, the method can include identifying, by the one or more computing devices, one or more related studies from the studies associated with the patient, each related study being associated with the first study. The method can include modifying, by the one or more computing devices and on the GUI, the appearance of the year icon associated with each year associated with a related study, respectively.

In accordance with the disclosed subject matter, a system for displaying a list of studies associated with a patient is provided. The system can include a workstation including at least a graphical user interface, one or more processors operationally coupled to the graphical user interface, and a memory operationally coupled to the processors and storing instructions executable by the processors. The processors can be operable when executing the instructions to: receive a request to view two or more studies associated with a patient; display, on the graphical user interface, a list of studies associated with the patient, the list including a study icon associated with each study; receive a user selection of a first study from the studies associated with the patient; modify, on the graphical user interface, the appearance of a first study icon associated with the first study; display, on the graphical user interface, at least one image associated with the first study; identify one or more related studies from the studies associated with the patient, each related study being associated with the first study; and modify, on the graphical user interface, the appearance of at least one study icon associated with one of the one or more related studies.

In accordance with the disclosed subject matter, a system for displaying a list of studies associated with a patient is provided. The system can include a workstation including at least a graphical user interface, one or more processors operationally coupled to the graphical user interface, and a memory operationally coupled to the processors and storing instructions executable by the processors. The processors can be operable when executing the instructions to: receive a request to view two or more studies associated with the patient, each study being associated with a year; display, on the graphical user interface, a scrollable list of the studies associated with the patient, the scrollable list including a study icon associated with each study; identify a beginning year, the beginning year being an earliest year associated with the studies; identify an ending year, the ending year being a latest year associated with the studies; and display, on the graphical user interface, a non-scrollable timeline, the non-scrollable timeline including a year icon associated with each year including and between the beginning year and the ending year.

In accordance with the disclosed subject matter one or more computer-readable non-transitory storage media embodying software are provided. The software can be operable when executed to: receive a request to view two or more studies associated with a patient; display, on a graphical user interface, a list of studies associated with the patient, the list including a study icon associated with each study; receive a user selection of a first study from the studies associated with the patient; modify, on the graphical user interface, the appearance of a first study icon associated with the first study; display, on the graphical user interface, at least one image associated with the first study; identify one or more related studies from the studies associated with the patient, each related study being associated with the first study; and modify, on the graphical user interface, the appearance of at least one study icon associated with one of the one or more related studies.

In accordance with the disclosed subject matter one or more computer-readable non-transitory storage media embodying software are provided. The software can be operable when executed to: receive a request to view two or more studies associated with the patient, each study being associated with a year; display, on a graphical user interface, a scrollable list of the studies associated with the patient, the scrollable list including a study icon associated with each study; identify a beginning year, the beginning year being an earliest year associated with the studies; identify an ending year, the ending year being a latest year associated with the studies; and display, on the graphical user interface, a non-scrollable timeline, the non-scrollable timeline including a year icon associated with each year including and between the beginning year and the ending year.

### DRAWINGS

FIG. 1 shows a hierarchy of medical image records that can be viewed in accordance with the disclosed subject matter.

FIG. 2A shows an exemplary GUI including a list of studies in accordance with the disclosed subject matter.

FIG. 2B shows the exemplary GUI including a list of studies and a timeline in accordance with the disclosed subject matter.

FIG. 3 shows the exemplary GUI including a list of studies with a selected study and related studies highlighted, in accordance with the disclosed subject matter.

FIG. 4 shows the exemplary GUI including a list of studies filtered for the selected study and related studies, in accordance with the disclosed subject matter.

FIG. 5 shows the exemplary GUI including a list of studies and a timeline in accordance with the disclosed subject matter.

FIGS. 6-8 show the exemplary GUI including a detailed timeline in accordance with the disclosed subject matter.

FIG. 9 shows the architecture of a system for displaying studies in accordance with the disclosed subject matter.

FIGS. 10 and 11 are a flow charts of methods for displaying a list of studies associated with a patient in accordance with the disclosed subject matter.

### DETAILED DESRIPTION

Reference will now be made in detail to the various exemplary embodiments of the disclosed subject matter, exemplary embodiments of which are illustrated in the accompanying drawings. Herein, the terms “study” and “medical study” can refer broadly to a compilation of medical information that can include a single medical image or a medical image record, or can include one or more of a medical image or a medical image record. The methods and systems described herein can be used for identifying and displaying associations and timelines of medical studies, such as medical studies stored on a picture archiving and communication system (PACS). A variety of records are suitable for the methods and systems of the present disclosure and the records can be stored in any system, for example a Vendor Neutral Archive (VNA). As used in the description and the appended claims, the singular forms, such as “a,” “an,” “the,” and singular nouns, are intended to include the plural forms as well, unless the context clearly indicates otherwise.

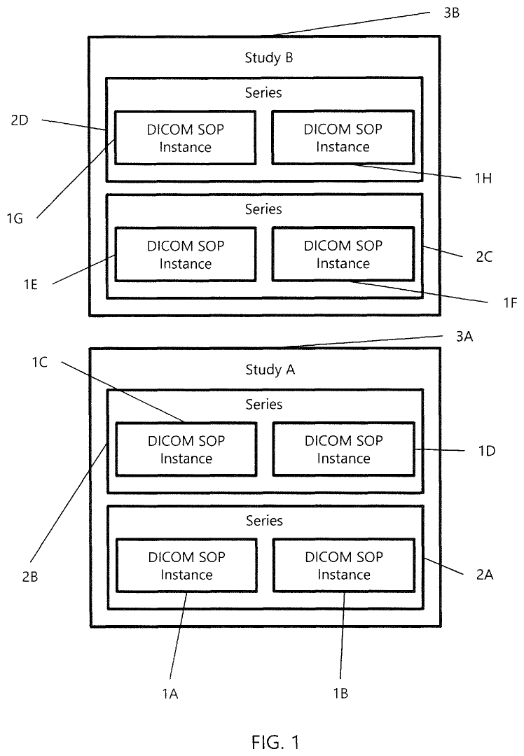

For purpose of illustration and not limitation, the systems and methods are described herein with respect to displaying Digital Imaging and Communications in Medicine (DICOM) records, stored on a PACS system. For example, and with reference to FIG. 1 for purpose of illustration and not limitation, as referred to herein a medical image record can include a single DICOM Service-Object Pair (“SOP”) Instance (also referred to as “DICOM Instance,” “DICOM image,” and “image”) 1 (e.g., 1A-1H), one or more DICOM SOP Instances 1 in one or more Series 2 (e.g., 2A-D), one or more Series 2 in one or more Studies 3 (e.g., 3A, 3B), and one or more Studies 3.

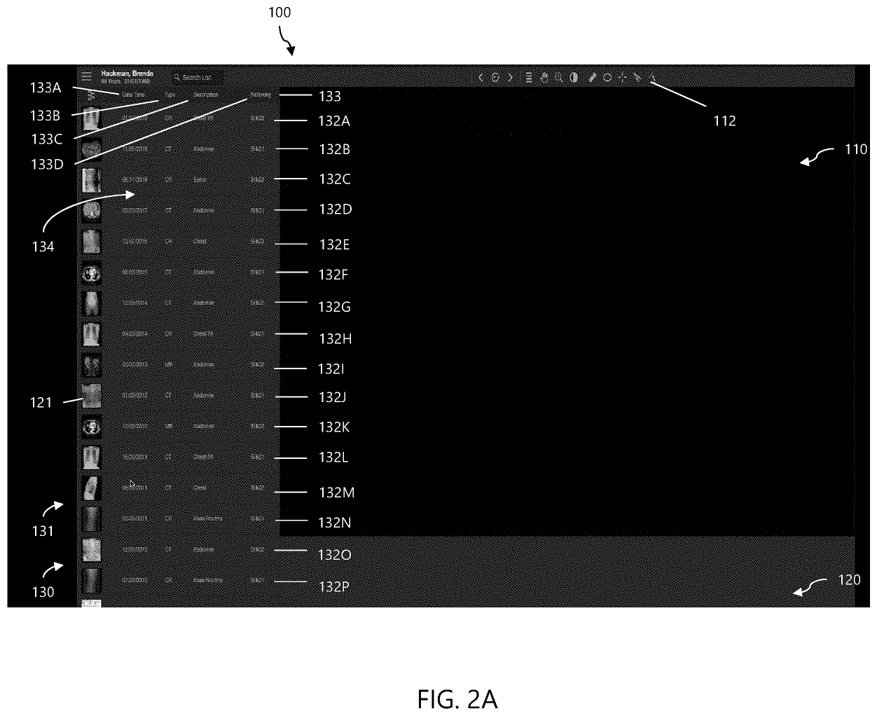

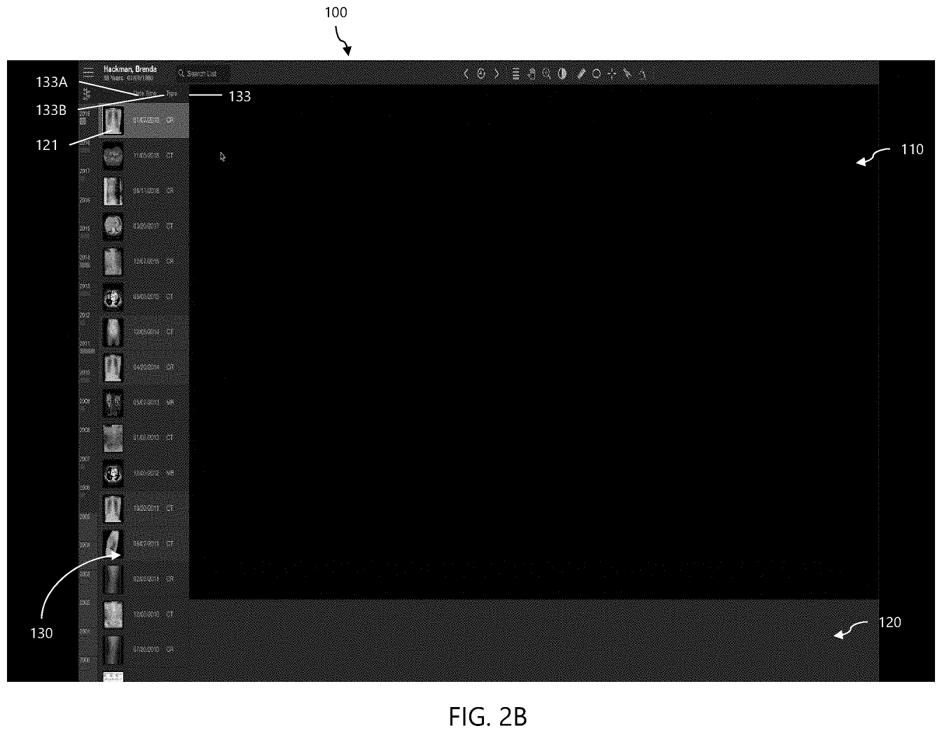

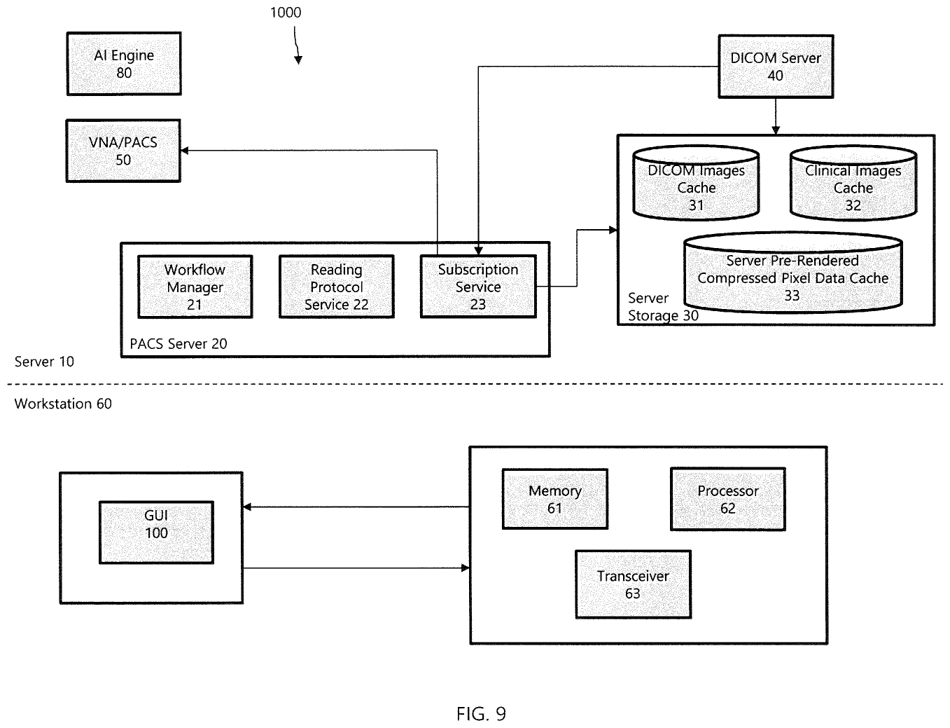

Referring to FIGS. 2-8 for purpose of illustration and not limitation, the disclosed methods and systems can include GUI 100. The GUI 100 can be displayed on a workstation 60 and can be controlled by one or more computing devices which can be on the workstation 60, server 10, or both (see FIG. 9). The GUI 100 can include an image display area 110, a thumbnail display area 120, and a study-list display area 130. The image display area 110 can include a tool bar 112 having various buttons for modifying or manipulating image 111. The tool bar 112 can be displayed, for example, along the top of the image display area 110.

Upon receiving a request from a user (for example a doctor, nurse, or other medical technician) to view studies associated with a particular patient, a list 131 of the studies associated with the patient can be displayed in the study-list display area 130. The studies can be DICOM Studies 3. The list 131 can include a study icon 132 (e.g., 132A-132P) for each Study 3 (e.g., 3A-3P) associated with the patient. The study list 131 can be scrollable, for example, up and down, if additional space is needed to include a study icon 132 for each Study 3 associated with the patient. The study-list display area 130 can also include a header row 133 with labels for the type of information 134 displayed in each study icon 132. For example, header row 133 can include a label for date 133A, which can refer to the date of the Study 3, type 133B, which can refer to the imaging modality of the Study 3, description 133C, which can refer to the anatomical structure that is the focus of the Study 3, and referring 133D, which can refer to the referring physician of the Study 3. Each study icon 132 can include the information 134. For example, as shown in FIG. 2A, study icon 132A which is associated with Study 3A displays that Study 3A occurred on January 7, 2019, is a CR-type Study, focuses on the chest posterior/anterior (PA) view of the patient, and was referred by the physician with the code Sik02. The study icon 132 can also include a thumbnail 121 of the Study 3. The thumbnail 121 can be based on at least one DICOM SOP Instance 1 in Study 3. The type of information 134 displayed for each Study 3 can be selected by the user, for example, by right clicking on the header row 133 and selecting from a list of information types. The information type can include date/time, type (modality or non-DICOM), description, referring physician, visit number, accession number, source (such as data source), or other relevant information types. In particular embodiments, a greater or fewer number of information columns can be used, and the width of the study-list display area 130 can be modified accordingly. With reference to FIG. 2B, for purpose illustration and not limitation, the user has selected to display only the thumbnail 121, date, and study type. Accordingly, header row 133 only includes labels for the date 133A and the type 133B, and the width of the study-list display area 130 is reduced.

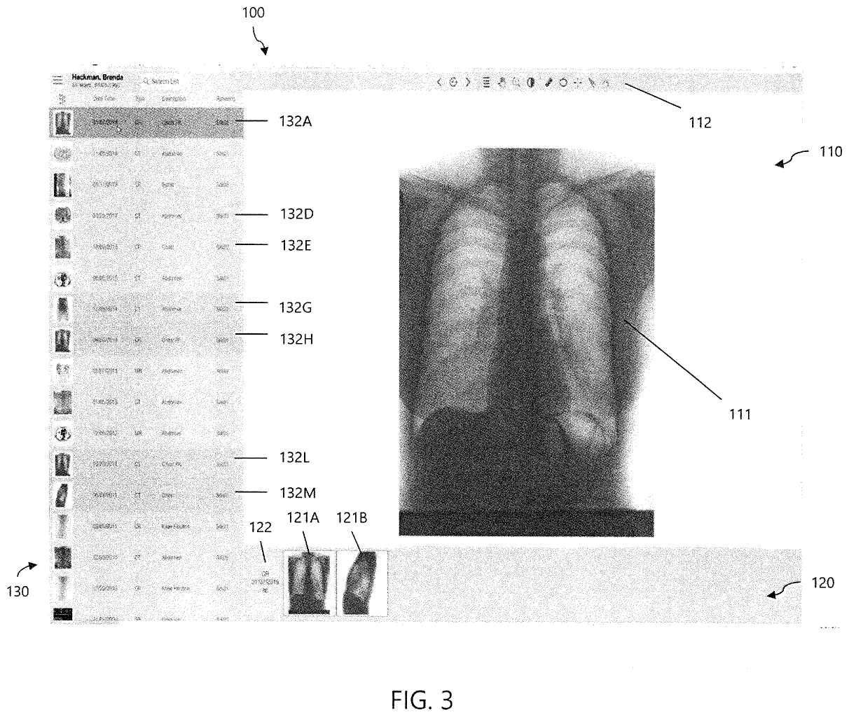

In accordance with the disclosed subject matter, a request can be received to display a particular Study 3. For example, a user can click (or otherwise select) a particular study icon 132. With reference to FIG. 3, for purpose illustration and not limitation, the user has clicked study icon 132A, which is associated with Study 3A. Upon receiving the request, the appearance of the selected study icon 132A can be modified. For example, the color of the study icon 132A can be changed, such as by highlighting study icon 132A in green. Additionally, or alternatively, the font in study icon 132A can be changed, the background of the study icon 132A can be changed, border properties of study icon 132A can be changed, the size of study icon 132A can be changed (for example, making the study icon 132A wider and/or taller), or other modifications can be made to study icon 132A to distinguish the selected study icon 132A from the non-selected study icons 132B-P. As an example and not by way of limitation, the border can be changed by making the border five times wider or changing the color of the border. Changes to the border can be made around the entire study icon 132A or limited to one or more edges of the study icon 132A. Although this disclosure described modifying the appearance of the study icons 132 in a particular manner, this disclosure contemplates modifying the appearance of the study icons 132 in any suitable manner.

Upon receiving the request, the image display area 110 can display an image 111. The image 111 can belong to a Study 3, for example, the image 111 can be a single DICOM SOP Instance 1 in the Study 3A. The image 111 can be any type of electronic medical image, such as x-rays (or x-rays' digital counterparts: CR and DR), mammograms, CT scans, MRIs, positron emission tomography PET scans and ultrasound images. As shown in FIG. 3, for purpose of illustration and not limitation, image 111 can be a CR image of a patient. The thumbnail display area 120 can include thumbnails 121, for example, thumbnails 121A, 121B. The thumbnail display 120 can include a thumbnail 121 for some or each DICOM SOP Instance 1 in the Study 3 associated with a selected study icon 132. The thumbnail display area 120 can be scrollable, for example to the left and right, if additional space is needed to display thumbnails 121. A thumbnail header 122 can provide information 134 regarding the Study 3 (i.e., the selected study).

In accordance with the disclosed subject matter, upon receiving a request to display a particular Study 3A, the system 1000 can identify one or more related Studies 3. The related Studies 3 can be associated with the selected Study 3A. Identifying related Studies 3 can be based on one or more factors. For example, identifying related Studies 3 can be based on a procedure code, such as the International Statistical Classification of Diseases and Related Health Problems (ICD), Healthcare Common Procedure Coding System (HCPCS), Current Procedural Terminology (CPT), Code on Dental Procedures and Nomenclature (CDT), or National Drug Codes (NDC), associated with the Studies 3. Studies 3 with the same or similar procedure codes can be identified as related. As an example and not by way of limitation, if a first Study 3 had a CPT code of 72100, which is the code for “Radiologic examination, spine, lumbosacral; 2 or 3 views” and a second Study 3 had a CPT code of 72110, which is the code for “Radiologic examination, spine, lumbosacral; minimum of 4 views,” the two Studies 3 can be identified as being related because the CPT codes are related. Additionally or alternatively, the association can be based on the anatomy in each Study 3. As an example and not by way of limitation, if a first Study 3 includes a CR image of a patient's chest, and a second Study 3 includes a CT scan of a patient's chest, the two studies can be identified as being related because both Studies 3 are focused on the patient's chest. Additionally or alternatively, the association can be based on the referring physician, for example, if the same physician or physicians in a particular practice have referred two Studies 3, the Studies 3 can be identified as being related. Additionally or alternatively the association can be based on a manual input by a user. For example, a user can choose (for example, right click) a Study 3 and identify it as associated (or not associated) with the selected Study 3. Additionally or alternatively, the association can be based on a machine learning process. If a physician manually indicates that a Study 3 is associated with another, machine learning can look for similarities among other similar Studies 3 that were also manually associated and begin associating based on those similarities. For example, and not by way of limitation, a machine learning algorithm can identify that Studies 3 with certain CPT codes have repeatedly been marked as related (for example, by manual user input), and moving forward the system 1000 can identify Studies 3 with the respective CPT codes as related. As another example, a machine learning algorithm can identify that Studies 3 that have been referred by certain physicians have been marked as related (for example, by manual user input), and moving forward the system 1000 can identify Studies 3 referred by those physicians as related. Machine learning can integrate various information types, such as imaging modality and anatomy. As an example, a machine learning algorithm can identify that Studies 3 with a certain CPT code are typically associated with certain anatomy and modality Studies 3 (e.g., chest CT), and moving forward the system 1000 can associate Studies 3 with the particular CPT codes, anatomy, and/or modalities. Additionally, or alternatively, machine learning can integrate patient demographic data. As an example, a machine learning algorithm can identify that all patients of a certain gender (e.g., male) and over a certain age (e.g., 75) having a particular procedure (e.g., angiogram) have certain other Studies 3 with a particular procedure code (e.g., Studies 3 with a chest CT procedure code) associated with the angiogram. Moving forward the system 1000 can make the learned associations for the particular patient population. Although this disclosure described identifying associated Studies 3 in a particular manner, this disclosure contemplates identifying associated Studies 3 in any suitable manner.

In accordance with the disclosed subject matter, the study icons 132 associated with related Studies 3 can be modified on GUI 100. With reference to FIG. 3, for purpose of illustration and not limitation, Studies 3G, 3H, 3L, and 3M have been identified as related to Study 3A. As noted above, Study 3A occurred on Jan. 7, 2019, is a CR-type Study, focuses on the chest PA of the patient, and was referred by the physician with the code Sik02. In FIG. 3, Study 3H was identified as related because it is the same study type (CR-type Study) and focuses on the same anatomy (chest PA). Study 3L was identified as related because it focuses on the same anatomy (chest PA). Study 3M was identified as related because it focuses on a similar anatomy (chest). Study 3G was identified as related by manual user input. Accordingly, the appearances of study icons 132G, 132H, 132L, and 132M associated with Studies 3G, 3H, 3L, and 3M, respectively, have been modified. By contrast, Studies 3D, 3E have not been identified as related to Study 3A. Study 3D includes a different study type (CT-type Study), focuses on different anatomy (abdomen) and was referred by a different physician (code Sik01). Study 3E has the same study type (CR-type Study) and a similar anatomy (chest) but was manually marked as not related by a user. Thus, the appearances of study icons 132D, 132E are not modified. In particular embodiments, the workstation 60 can further download all DICOM SOP Instances 1 associated with the related Studies 3G, 3H, 3L, 3M and store the DICOM SOP Instances I at the workstation 60 for quick viewing. More information on downloading and rendering medical images can be found in U.S. application Ser. No. 16/450,477, which is incorporated by reference herein.

The study icons 132G, 132H, 132L, 132M can be modified by highlighting, for example in green, but can include a different (e.g., duller) color than selected study icon 132A. This can allow the user to see what Studies 3 in the patient's overall history are clinically relevant to the current Study 3A being evaluated without having to sift through every patient record. For example, if a physician is reviewing Study 3A and focusing on a carcinoma in a patient, the physician can easily identify related Studies 3G, 3H, 3L, 3M that will provide insight into the carcinoma, for example, how it has changed in size over time in a number of different Studies 3A, 3G, 3H, 3L, 3M. Although the study icons 132G, 132H, 132L, 132M are illustrated as modified by highlighting, the study icons 132G, 132H, 132L, 132M can be modified by changing font in study icons 132G, 132H, 132L, 132M, changing the size of study icons 132G, 132H, 132L, 132M, changing the background of study icons 132G, 132H, 132L, 132M, changing the border properties of study icons 132G, 132H, 132L, 132M, or other modifications can be made to study icons 132G, 132H, 132L, 132M, to distinguish the associated study icons 132G, 132H, 132L, 132M, from the non-associated study icons (e.g., 132G, 132E) and the selected study icon 132A.

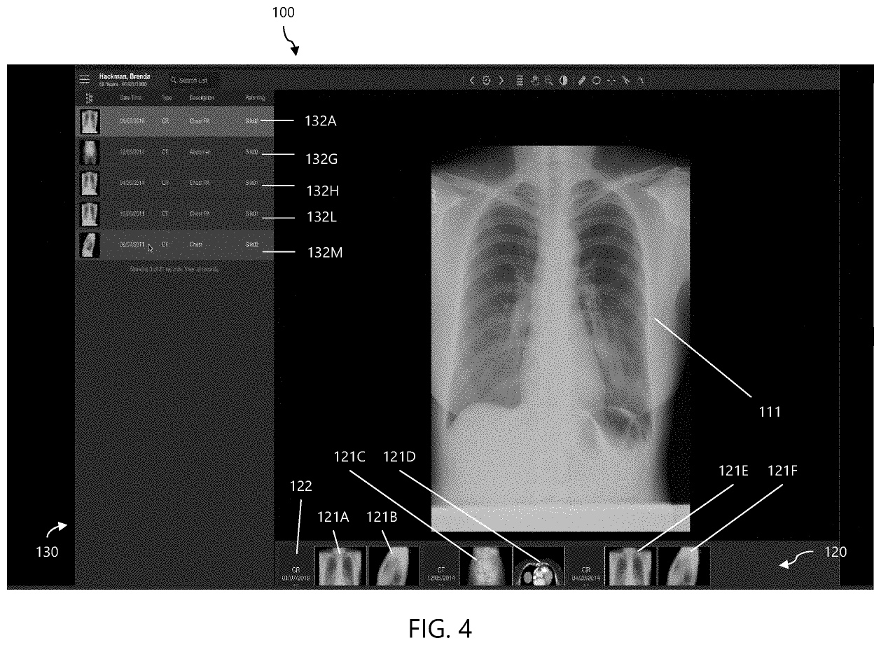

With reference to FIG. 4, for purpose of illustration and not limitation, the user can filter the study icons 132 in the study list 131 to only show the study icon 132A associated with the selected Study 1A and the study icons 132G, 132H, 132L, 132M associated with the related Studies 3G, 3H, 3L, 3M. That is, study icons (e.g., 132D, 132E) associated with non-related Studies (e.g., 3D, 3E) can be removed from the study list 131. The user can filter the study icons 132, for example, by right clicking on the study list 131 and selecting a filter option. The user can further filter the study icons 132 in the study list 131, for example, by date/time, type (modality or non-DICOM), description, referring physician, visit number, accession number, source (such as data source), or other relevant information types. Additionally or alternatively, the user can set his or her preferences such that certain filters are applied automatically when the user logs into workstation 60, launches GUI 100, and/or accesses Studies 3 associated with a particular patient. Additionally or alternatively, the user can select to display thumbnails 121 associated with each of the selected and related Studies 3A, 3G, 3H, 3L, 3M in the thumbnail display area 120. FIG. 4 shows thumbnails 121A, 121B from selected Study 3A, thumbnails 121C, 121D from related Study 3G, and thumbnails 121E, 121F from related Study 3H displayed in the thumbnail display area 120. Information 134 about each respective Study 3 is provided in thumbnail headers 122.

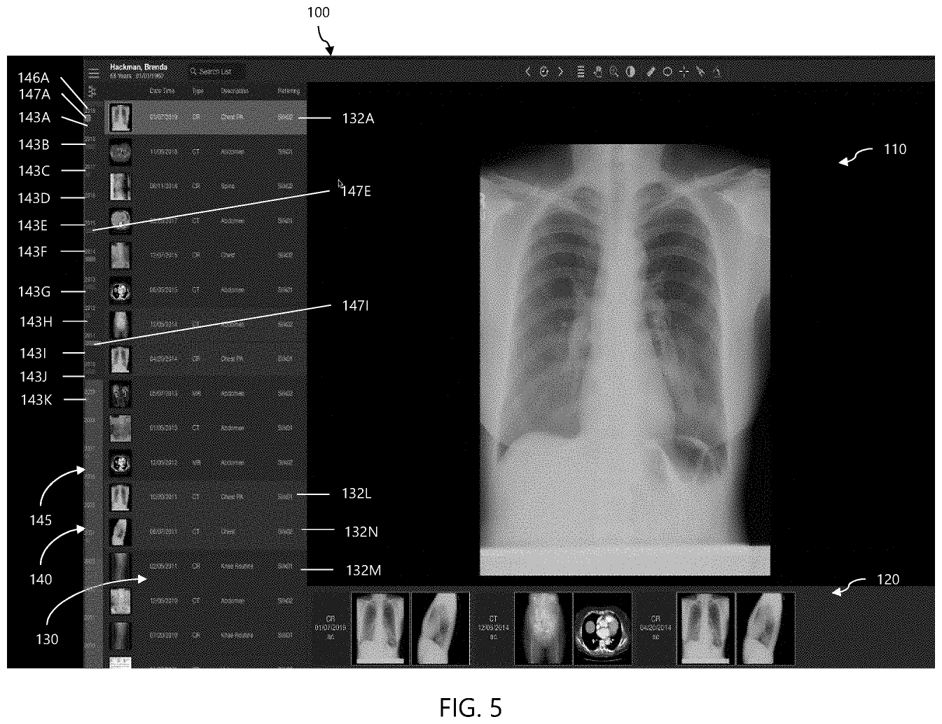

With reference to FIG. 5, for purpose of illustration and not limitation, the GUI 100 can include a timeline display area 140 to display a timeline 145. Upon receiving the request to view Studies 3 associated with a particular patient, the system 1000 can identify a beginning year 141 and an ending year 142. The beginning year 141 can be the earliest year associated with the Studies 3 and the ending year 142 can be the latest year associated with the Studies 3. For example, if the earliest Study 3 associated with the patient occurred in 2000, the beginning year 141 can be the year 2000. If the most recent Study 3 associated with the patient occurred in the year 2019, the ending year 142 can be the year 2019. The timeline 145 can include a year icon 143 associated with each year between (and including) the beginning year 141 and the ending year 142. As shown in FIG. 5, for purpose of illustration and not limitation, the beginning year 142 is the year 2000 and the ending year 142 is the year 2019. Accordingly the timeline 145 includes 20 year icons 143 (e.g., 143A, 143B) associated with each year between (and including) the year 2000 and the year 2019. Each year can be included regardless of whether there are Studies 3 associated with a given year. The timeline 145 can be non-scrollable. Accordingly, the size (for example, the height) of the year icons 143 can be determined based on the number of year icons 143 to be included in the timeline 145 and the size of the timeline display area 140. For example, if 10 year icons 143 need to be displayed, the icons can include a larger height than if 20 year icons 143 need to be displayed. This can allow the user to see every single year back to the beginning year 141 without scrolling. The timeline 145 can be automatically resized if the size of the GUI 100 (or timeline display area 140) is adjusted. Furthermore, with no years hidden (i.e., because there is a year icon 143 for every year), the user can quickly see and understand a patient's true clinical timeline. If the number of years between the beginning year 141 and the ending year 142 is below a threshold or within a range (e.g., two to five years), additional year icons 143 can be provided. For example, a minimum of 5 year icons 143 can be created. As another example, if the number of years between the beginning year 141 and the ending year 142 is below a threshold (e.g., two years), timeline 145 can display month icons instead of year icons 143. The month icons can have each feature of the year icons 143 described above and below and can display the relevant month and year (e.g., [Jan '19], [Feb '19], [Mar '19], etc.). As another example, quarter icons, which can represent three months, can be used. The quarter icons can have each feature of the year icons 143 described above and below and can display the relevant quarter and year (e.g., [Jan '19], [Apr '19], [July '19], [Oct '19]).

In accordance with the disclosed subject matter, each year icon 143 can include a year label 146. The year label 146 can indicate which year the year icon 143 is associated with. For example, and as shown in FIG. 5, year icon 143A is associated with the year 2019, and therefore year label 146A identifies year 2019. Additionally or alternatively, study bars 147 can be provide for each year that contains a Study 3. For example, and as shown in FIG. 5, year icon 143A includes study bar 147A, year icon 143E includes study bar 147E, and year icon 1431 includes study bar 1471. No Studies 3 are associated with the year 2016, and accordingly year icon 143 does not include a study bar 147. The size (e.g., the width and/or length) of the study bar 147 can be detet mined by the quantity of Studies 3 associated with the year. The year with the most Studies 3 can have a study bar 147 with the maximum length, and can act as a baseline for other years. For example, and as shown in FIG. 5, there are three Studies 3 in 2011 (the most of any year), and accordingly study bar 1471 is the largest. By contrast, there are two Studies 3 2015 and one Study 3 in 2019, and the study bars 147A, 147E have been scaled accordingly. That is, study bar 147E is two thirds the size of study bar 1471, and study bar 147A is one third the size of study bar 1471. In particular embodiments, the number of Studies 3 associated with the year can be displayed in the respective study bar 147. Additionally or alternatively, the associated year and/or number of Studies 3 can be provided if the user hovers over the respective year icon 143 and/or study bar 147. Additionally or alternatively, additional information 134 about the Study 3 or Studies 3 associated with a particular year can be displayed if the user hovers over the respective year icon 143 and/or study bar 147. For example, GUI 100 can display date/time, type (modality or non-DICOM), description, referring physician, visit number, accession number, source (such as data source), number of images within the Study 3 or other relevant information 134. The displayed information 134 can be broken down for each Study 3 associated with the year (e.g., with each Study's 3 information 134 displayed in a row, with a total of three rows if there are three Studies 3 associated with the year).

In accordance with the disclosed subject matter, upon receiving a request to display a particular Study 3, the appearance of the corresponding year icon 143 and/or study bar 147 can be modified. With reference to FIG. 5, for purpose of illustration and not limitation, the user has selected study icon 132A, which occurred in the year 2019. Accordingly, study bar 147A has been modified by highlighting study bar 147A in green and adding a white border. Additionally or alternatively, the appearance of year icons 143 and/or study bars 147 for years including Studies 3 related to Study 3A can be modified. For example, the year 2011 includes related studies 3L, 3M, and accordingly study bar 1471, is highlighted in green. The year 2015 does not include related studies, and accordingly the appearance of study bar 147E is not modified. Additionally or alternatively, the appearance of the year icons 143 can be modified based on the study icons 132 that are visible in the study-list display area 130. For example, FIG. 5 shows study icons 132 corresponding to Studies 3 ranging from 2019 (the ending year 142) to the year 2010. Accordingly the appearance of year icons 143 associated with the year 2009 (e.g., 143K) and earlier have been modified by making those year icons 143 slightly darker (or duller). Although this disclosure described modifying the appearance of the year icons 143 and/or study bars 147 in a particular manner, this disclosure contemplates modifying the appearance of the year icons 143 and/or study bars 147 in any suitable manner.

In accordance with the disclosed subject matter, the user can navigate the list 131 by interacting with the timeline 145. For example, and not by way of limitation, a user can automatically scroll the timeline 145 to a specific year by clicking on the respective year icon 143. If a user clicks on a specific year icon 143 (e.g., 1431), the list 131 can automatically scroll to move study icons 132L, 132M, 132N associated with 2011 to the top of the visible portion of the list study-list display area 130. Additionally or alternatively, a user can click a study bar 147 to filter the list 131 to display only the study icons 132 associated with Studies 3 from the respective year. That is, if a user clicks study bar 1471, the list 131 can be filed to display only study icons 132N, 132N, 132M. A message can be displayed at the bottom informing the user that other years have been filtered out of the study-list display area 130. Although this disclosure described navigating the list 131 using the year icons 143 in a particular manner, this disclosure contemplates navigating the list 131 using the year icons 143 in any suitable manner.

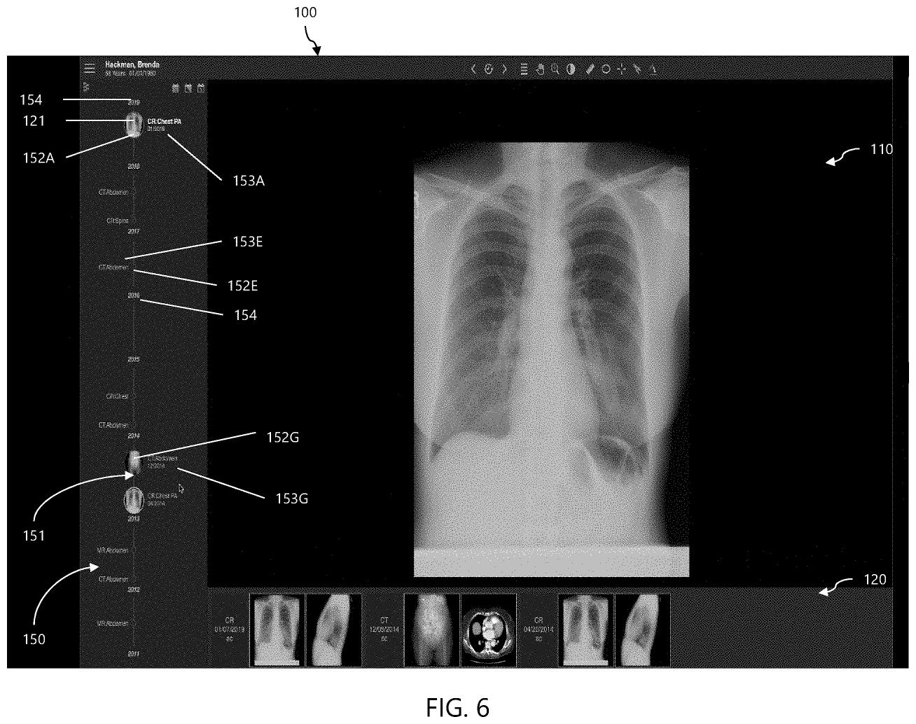

With reference to FIGS. 6-8, for purpose of illustration and not limitation, GUI 100 can include a detailed-timeline display area 150 to display a detailed timeline 151. The detailed timeline 151 can include a study bubble 152 and bubble label 153, each associated with a Study 3. The bubble label 153 can include information 134 about the respective Study 3. For example, bubble label 153E notes that Study 3E is a CT-type study of the abdomen. The detailed timeline 151 can also include a year label 154.

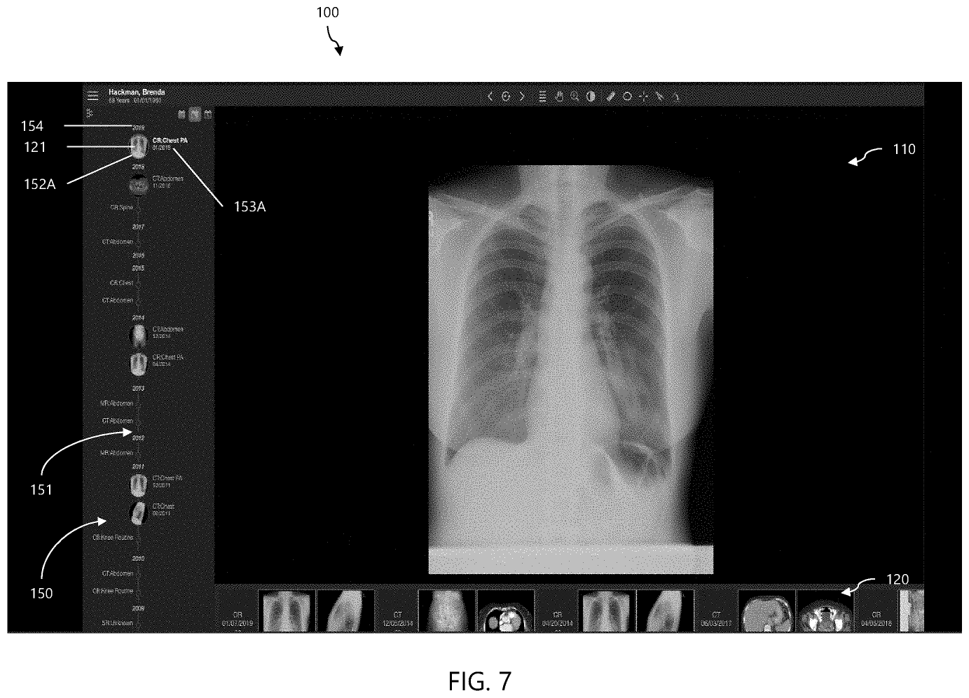

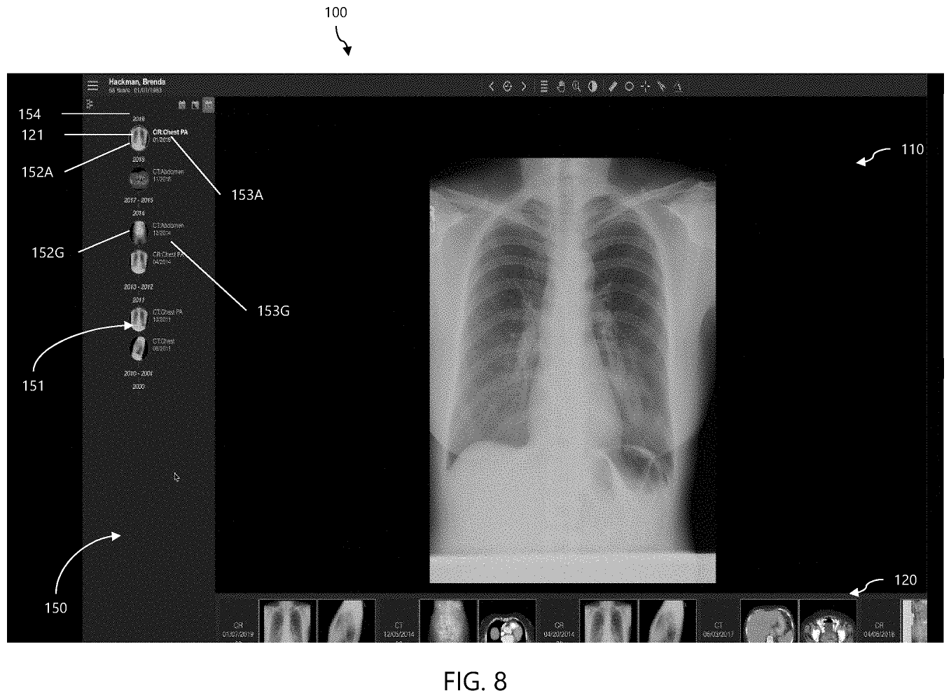

In accordance with the disclosed subject matter, upon receiving a request to display a particular Study 3, the appearance of the corresponding study bubble 152 and bubble label 153 can be modified. With reference to FIG. 8, for purpose of illustration and not limitation, the user has selected to view Study 3A, by selecting study bubble 152A. Accordingly, study bubble 152A has increased in size, a green highlighting ring has been added around study bubble 152A, and a thumbnail 121 is shown inside study bubble 152A. Furthermore, additional information is provided in bubble label 153A, particularly that the date of the Study 3A is January 2019. Additionally or alternatively, the appearance of study bubbles 152 and/or bubble labels 153 for years including Studies 3 related selected Study 3A can be modified. For example, study bubble 152G and bubble label 153G, which correspond with related Study 3G, are modified. Study bubble 152G is increased in size and a thumbnail 121 is shown in study bubble 152G. Furthermore, bubble label 153G includes additional information, particularly the date of Study 3G. With reference to FIG. 7, for purpose illustration and not limitation, the space between study bubbles 152 can be modified, for example, the space can be reduced. With reference to FIG. 8, for purpose of illustration and not limitation, the user can filter the study bubbles 152, for example, by right clicking on the detailed timeline 152 and selecting a filter option. For example, the user can filter the study bubbles 152 in the detailed timeline 151 to only show the study bubble 152 associated with the selected Study 1A and the study bubbles (e.g., 152G) associated with the related Studies (e.g., 3G).

An exemplary architecture of a system 1000 for display of medical images and information, in accordance with the disclosed subject matter, is provided in FIG. 9 for purpose of illustration and not limitation. The system 1000 can include one or more computing devices defining a server side 10 and a user workstation 60 coupled to one or more computing devices by a network. The computing device, for example, can be a server or client device, while the network, for example, can be a Local Area Network (“LAN”), a Wireless LAN (“WLAN”), a virtual private network (“VPN”), or any other network that allows for any radio frequency or wireless type connection. For example, other radio frequency or wireless connections can include, but are not limited to, one or more network access technologies, such as Global System for Mobile communication (“GSM”), Universal Mobile Telecommunications System (“UMTS”), General Packet Radio Services (“GPRS”), Enhanced Data GSM Environment (“EDGE”), Third Generation Partnership Project (“3GPP”) Technology, including Long Term Evolution (“LTE”), LTE-Advanced, 3G technology, Internet of Things (“IOT”), fifth generation (“5G”), or new radio (“NR”) technology. Other examples can include Wideband Code Division Multiple Access (“WCDMA”), Bluetooth, IEEE 802.11b/g/n, or any other 802.11 protocol, or any other wired or wireless connection.

System 1000 can be configured to ensure one or more medical images or medical image records are transferred from server 10 to workstation 60. The one or more medical images or records can be cached in workstation 60 before a user opens the images or records at workstation.

A user can be any person authorized to access workstation 60, including any health professional, medical technician, or a patient. In some embodiments a user authorized to communicate with the PACS can have a username and/or password that can be used to login or access workstation 60. System 1000 can help to provide an improved reading experience in all network environments, including when workstation 60 is accessed on-premises or remotely.

In accordance with the disclosed subject matter, server 10 can include a PACS server module 20, a server storage 30, a DICOM server 40, and/or an additional data source, such as a VNA/PACS 50, remote PACS, VNA, or another vendor PACS/VNA. Workstation 60 can include a GUI 100, memory or storage 61, processor 62, and/or transceiver 63.

Workstation 60 can receive one or more images or records from server 10 using transceiver 63. Transceiver 63 can, independently, be a transmitter, a receiver, or both a transmitter and a receiver, or a unit or device that can be configured both for transmission and reception. In other words, transceiver 63 can include any hardware or software that allow workstation 60 to communicate with server 10 and/or AI engine 80. Transceiver 63 can be either a wired or a wireless transceiver. When wireless, the transceiver, in certain embodiments, can be implemented as a remote radio head which is not located in the device itself, but in a mast. While FIG. 9 only illustrates a single transceiver, workstation 60 can include one or more transceivers.

The images received by workstation 60 can be processed using one or more processors 62. Processor 62 can be any hardware or software used to execute computer program instructions. These computer program instructions can be provided to a processor of a general purpose computer to alter its function to a special purpose, a special purpose computer, application-specific integrated circuit (“ASIC”), or other programmable digital data processing apparatus, such that the instructions, which execute via the processor of the client station or other programmable data processing apparatus, implement the functions/acts specified in the block diagrams or operational block or blocks, thereby transforming their functionality in accordance with embodiments herein. In certain non-limiting embodiments, the processor can be a portable embedded micro-controller or micro-computer. For example, processor 62 can be embodied by any computational or data processing device, such as a central processing unit (“CPU”), digital signal processor (“DSP”), application specific integrated circuit (“ASIC”), programmable logic devices (“PLDs”), field programmable gate arrays (“FPGAs”), digitally enhanced circuits, or comparable device or a combination thereof The processors can be implemented as a single controller, or a plurality of controllers or processors.

In certain embodiments, the records and/or images received at workstation 60 can be cached in memory 61 of workstation 60. Memory 61 can be a non-volatile storage medium or any other suitable storage device, such as a non-transitory computer-readable medium or storage medium. For example, memory 61 can be a random-access memory (“RAM”), read-only memory (“ROM”), hard disk drive (“HDD”), erasable programmable read-only memory (“EPROM”), electrically erasable programmable read-only memory (“EEPROM”), flash memory or other solid-state memory technology. Memory 61 can also be a compact disc read-only optical memory (“CD-ROM”), digital versatile disc (“DVD”), any other optical storage, magnetic cassettes, magnetic tape, magnetic disk storage or other magnetic storage devices, or any other physical or material medium which can be used to tangibly store the desired information or data or instructions and which can be accessed by a computer or processor. Memory 61 can be either removable or non-removable.

Workstation 60 can take the form of any known client device. For example, workstation 60 can be a computer, such as a laptop or desktop computer, a personal data or digital assistant (“PDA”). or any other user equipment or tablet, such as a mobile device or mobile portable media player. Server 10, on the other hand, can be a service point which provides processing, database, and communication facilities. For example, the server can include dedicated rack-mounted servers, desktop computers, laptop computers, set top boxes, integrated devices combining various features, such as two or more features of the foregoing devices, or the like. Servers can vary widely in configuration or capabilities, but can include one or more processors, memory, and/or transceivers. A server can also include one or more mass storage devices, one or more power supplies, one or more wired or wireless network interfaces, one or more input/output interfaces, and/or one or more operating systems.

Although FIG. 9 illustrates memory 61, processor 62, and/or transceiver 63 as being included in workstation 60, server 10 and AI engine 80 can similarly include a memory, a processor, and/or a transceiver. For example, server storage 30 shown in FIG. 1 can correspond to memory 61, while DICOM server 40 can correspond to processor 62.

In accordance with the disclosed subject matter, server 10 can include a PACS server 20 with workflow manager 21, reading protocol service 22, and/or subscription service 23.

Workflow manager 21, for example, can receive requests for records and/or images from workstation 60. Based on the user's subscription, subscription service 23 can coordinate with VNA/PACS 50, DICOM server 40, and server storage 30 to obtain the requested images and/or records. Reading protocol service 22 in PACS server 20 can also be used to identify prior records and/or images that have been stored by server 10. Protocol service 22, for example, can have access to a patient's history and can filter out non-relevant prior images or records based on the preference of the user.

Caching the images and/or records can include downloading one or more DICOM images and storing the downloaded images in memory 61 of workstation 60. Subscription service 23 can be used to identify the applicable DICOM images stored in server storage 30. Server storage 30 can include DICOM images cache 31, clinical images cache 32, and/or server pre-rendered and compressed pixel data cache 33. In certain embodiments one or more images or records can be stored in an external storage, such as VNA/PACS 50. In such embodiments, subscription service 23 can query the VNA/PACS 50 to obtain a list of DICOM images or records. The queries can be made using a DICOM messaging protocol, a Web Access for DICOM Objects (“WADO”) operation (“such as or WADO-RS”), or HyperText Transfer Protocol (“HTTP”).

When workstation 60 receives an image from server 10, the image can be rendered in any possible data form. For example, the image can be rendered as a Portable Network Graphics (“PNG”) file, used for higher quality viewing, as a Joint Photographic Experts Group (“JPEG”) file, used for lower quality viewing, as a Moving Picture Experts Group (“MPEG”) file, or any other image file format. In certain embodiments, the images may be stored, received, and/or transmitted at workstation 60 or server 10 in a compressed format. Compressed images, for example, can be cached in server 33 included in server storage 30. Once received, workstation 60 can decompress and display the image in GUI 100.

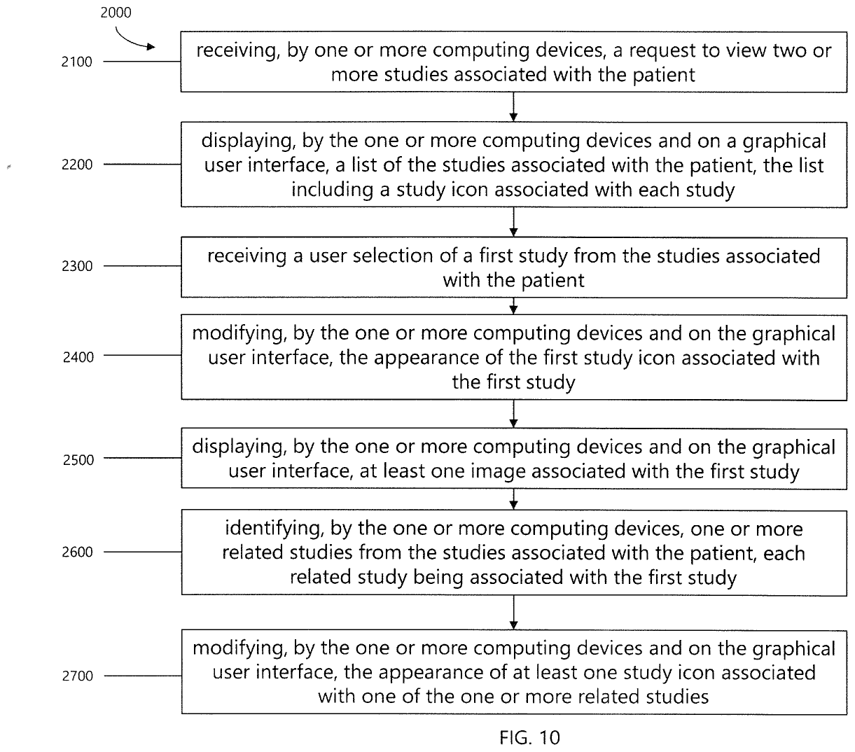

FIG. 10 illustrates an example method 2000 for displaying a list of studies associated with a patient. The method can begin at step 2100, where the method includes receiving, by one or more computing devices, a request to view two or more studies associated with the patient. At step 2200, the method can include displaying, by the one or more computing devices and on a GUI, a list of the studies associated with the patient, the list including a study icon associated with each study. At step 2300, the method can include receiving a user selection of a first study from the studies associated with the patient. At step 2400, the method can include modifying, by the one or more computing devices and on the graphical user interface, the appearance of a first study icon associated with the first study. At step 2500, the method can include displaying, by the one or more computing devices and on the GUI, at least one image associated with the first study. At step 2600, the method can include identifying, by the one or more computing devices, one or more related studies from the studies associated with the patient, each related study being associated with the first study. At step 2700, the method can include modifying, by the one or more computing devices and on the GUI, the appearance of at least one study icon associated with one of the one or more related studies. In accordance with the disclosed subject matter, the method can repeat one or more steps of the method of FIG. 10, where appropriate. Although this disclosure describes and illustrates particular steps of the method of FIG. 10 as occurring in a particular order, this disclosure contemplates any suitable steps of the method of FIG. 10 occurring in any suitable order. Moreover, although this disclosure describes and illustrates an example method for displaying a list of studies associated with a patient including the particular steps of the method of FIG. 10, this disclosure contemplates any suitable method for displaying a list of studies associated with a patient including any suitable steps, which can include all, some, or none of the steps of the method of FIG. 10, where appropriate. Furthermore, although this disclosure describes and illustrates particular components, devices, or systems carrying out particular steps of the method of FIG. 10, this disclosure contemplates any suitable combination of any suitable components, devices, or systems carrying out any suitable steps of the method of FIG. 10.

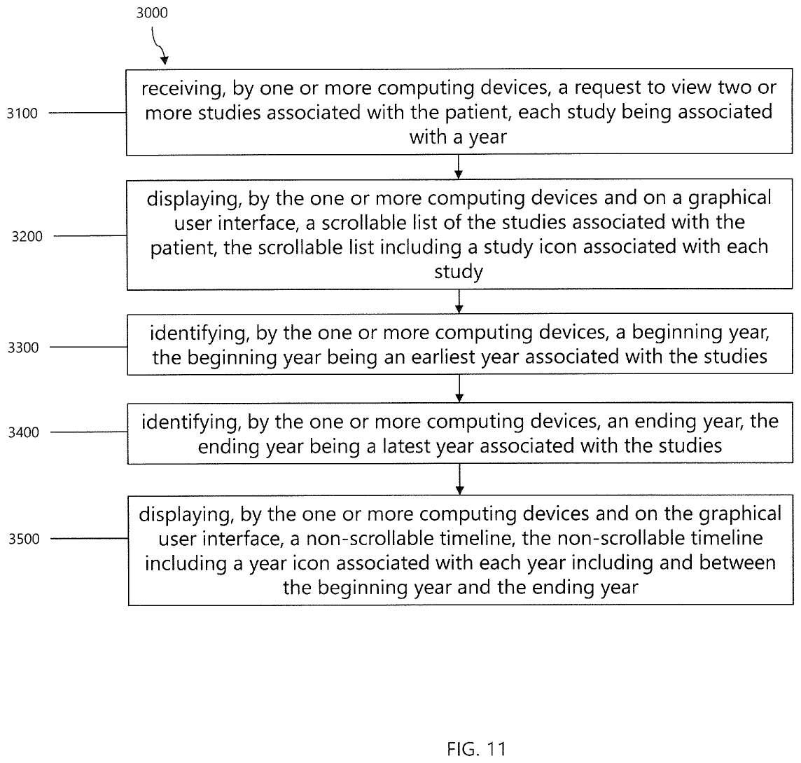

FIG. 11 illustrates an example method 3000 for displaying a list of studies associated with a patient. The method can begin at step 3100, where the method includes receiving, by one or more computing devices, a request to view two or more studies associated with the patient, each study being associated with a year. At step 3200, the method can include displaying, by the one or more computing devices and on a GUI, a scrollable list of the studies associated with the patient, the scrollable list including a study icon associated with each study. At step 3300, the method can include identifying, by the one or more computing devices, a beginning year, the beginning year being an earliest year associated with the studies. At step 3400, the method can include identifying, by the one or more computing devices, an ending year, the ending year being a latest year associated with the studies. At step 3500, the method can include displaying, by the one or more computing devices and on the GUI, a non-scrollable timeline, the non-scrollable timeline including a year icon associated with each year including and between the beginning year and the ending year. In accordance with the disclosed subject matter, the method can repeat one or more steps of the method of FIG. 11, where appropriate. Although this disclosure describes and illustrates particular steps of the method of FIG. 11 as occurring in a particular order, this disclosure contemplates any suitable steps of the method of FIG. 11 occurring in any suitable order. Moreover, although this disclosure describes and illustrates an example method for displaying a list of studies associated with a patient including the particular steps of the method of FIG. 11, this disclosure contemplates any suitable method for displaying a list of studies associated with a patient including any suitable steps, which can include all, some, or none of the steps of the method of FIG. 11, where appropriate. Furthermore, although this disclosure describes and illustrates particular components, devices, or systems carrying out particular steps of the method of FIG. 11, this disclosure contemplates any suitable combination of any suitable components, devices, or systems carrying out any suitable steps of the method of FIG. 11.

As described above in connection with certain embodiments, certain components, e.g., server 10 and workstation 60, can include a computer or computers, processor, network, mobile device, cluster, or other hardware to perform various functions. Moreover, certain elements of the disclosed subject matter can be embodied in computer readable code which can be stored on computer readable media and which when executed can cause a processor to perform certain functions described herein. In these embodiments, the computer and/or other hardware play a significant role in permitting the system and method for displaying medical image records. For example, the presence of the computers, processors, memory, storage, and networking hardware provides the ability to display medical image records in a more efficient manner. Moreover, the display of medical image records, cannot be accomplished with pen or paper, as such information is received over a network in electronic form.

The subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium also can be, or may be included in, one or more separate physical components or media (e.g., multiple CDs, disks, or other storage devices).

The term “processor” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program can include, by way of example and not by way of limitation, both general and special purpose microprocessors. Devices suitable for storing computer program instructions and data can include all forms of non-volatile memory, media and memory devices, including by way of example but not by way of limitation, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Additionally, as described above in connection with certain embodiments, certain components can communicate with certain other components, for example via a network, e.g., a local area network or the internet. To the extent not expressly stated above, the disclosed subject matter is intended to encompass both sides of each transaction, including transmitting and receiving. One of ordinary skill in the art will readily understand that with regard to the features described above, if one component transmits, sends, or otherwise makes available to another component, the other component will receive or acquire, whether expressly stated or not.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A method for displaying a list of studies associated with a patient, comprising:
receiving, by one or more computing devices, a request to view two or more studies associated with the patient;
displaying, by the one or more computing devices and on a graphical user interface, the list of the studies associated with the patient, the list including a study icon associated with each study;
receiving a user selection of a first study from the studies associated with the patient;
modifying, by the one or more computing devices and on the graphical user interface, an appearance of a first study icon associated with the first study;
displaying, by the one or more computing devices and on the graphical user interface, at least one image associated with the first study;
identifying, by the one or more computing devices and without additional user input, one or more related studies from the studies associated with the patient, each related study being associated with the first study; and
modifying, by the one or more computing devices and on the graphical user interface, an appearance of at least one study icon associated with one of the one or more related studies.

2. The method of claim 1, wherein each study icon displays information regarding the respective associated study.

3. The method of claim 2, wherein the displayed information includes one or more of a date, a modality, an anatomical structure, a referring physician, and a thumbnail of the study.

4. The method of claim 3, further comprising receiving a user request to change the displayed information; and
changing the displayed information based on the user request.

5. The method of claim 1, wherein identifying the one or more related studies from the studies associated with the patient is based at least in part on at least one of a procedure code associated with each study, an anatomical structure associated with each study, and a machine learning model.

6. A method for displaying a list of studies associated with a patient, comprising:
receiving, by one or more computing devices, a request to view two or more studies associated with the patient, each study being associated with a year;
displaying, by the one or more computing devices and on a graphical user interface, a scrollable list of the studies associated with the patient, the scrollable list including a study icon associated with each study;
identifying, by the one or more computing devices, a beginning year, the beginning year being an earliest year associated with the studies;
identifying, by the one or more computing devices, an ending year, the ending year being a latest year associated with the studies; and
displaying, by the one or more computing devices and on the graphical user interface, a non-scrollable timeline, the non-scrollable timeline including a year icon associated with each year including and between the beginning year and the ending year.

7. The method of claim 6, wherein each study icon displays information regarding the respective associated study.

8. The method of claim 7, wherein the information includes one or more of a date, a modality, an anatomical structure, a referring physician, and a thumbnail of the study.

9. The method of claim 8, further comprising receiving a user request to change the displayed information; and
changing the displayed information based on the user request.

10. The method of claim 6, wherein for each year that is associated with at least one study, the year icon associated with the respective year displays a number-of-studies indicator.

11. The method of claim 10, wherein the number-of-studies indicator is a bar having a length, the length being based on a number of studies associated with the respective year.

12. The method of claim 11, wherein the bar lengths are dynamically generated based on the number of studies associated with each year.

13. The method of claim 6, further comprising receiving a user selection of a first year icon associated with a selected year; and
scrolling the scrollable list of studies to display the study icons associated with the selected year at a top of the scrollable list.

14. The method of claim 6, further comprising receiving a user selection of a first study from the studies associated with the patient; and
modifying, by the one or more computing devices and on the graphical user interface, an appearance of the year icon associated with the year associated with the first study.

15. The method of claim 14, further comprising identifying, by the one or more computing devices, one or more related studies from the studies associated with the patient, each related study being associated with the first study.

16. The method of claim 15, further comprising modifying, by the one or more computing devices and on the graphical user interface, the appearance of the year icon associated with each year associated with a related study, respectively.

17. A system for displaying a list of studies associated with a patient, comprising:
a workstation including at least
a graphical user interface,
one or more processors operationally coupled to the graphical user interface, and
a memory operationally coupled to the processors and storing instructions executable by the processors;
wherein the processors are operable when executing the instructions to:
receive a request to view two or more studies associated with the patient;
display, on the graphical user interface, the list of studies associated with the patient, the list including a study icon associated with each study;
receive a user selection of a first study from the studies associated with the patient;
modify, on the graphical user interface, an appearance of a first study icon associated with the first study;
display, on the graphical user interface, at least one image associated with the first study;
identify, without additional user input, one or more related studies from the studies associated with the patient, each related study being associated with the first study; and
modify, on the graphical user interface, the an appearance of at least one study icon associated with one of the one or more related studies.

18. The system of claim 17, wherein each study icon displays information regarding the respective associated study.

19. The system of claim 18, wherein the displayed information includes one or more of a date, a modality, an anatomical structure, a referring physician, and a thumbnail of the study.

20. The system of claim 19, wherein the processors are further configured to receive a user request to change the displayed information; and
change the displayed information based on the user request.

21. The system of claim 17, wherein identifying the one or more related studies from the studies associated with the patient is based at least in part on at least one of a procedure code associated with each study, an anatomical structure associated with each study, and a machine learning model.

22. A system for displaying a list of studies associated with a patient, comprising:
a workstation including at least
a graphical user interface,
one or more processors operationally coupled to the graphical user interface, and
a memory operationally coupled to the processors and storing instructions executable by the processors;
wherein the processors are operable when executing the instructions to:
receive a request to view two or more studies associated with the patient, each study being associated with a year;
display, on the graphical user interface, a scrollable list of the studies associated with the patient, the scrollable list including a study icon associated with each study;
identify a beginning year, the beginning year being an earliest year associated with the studies;
identify an ending year, the ending year being a latest year associated with the studies; and
display, on the graphical user interface, a non-scrollable timeline, the non-scrollable timeline including a year icon associated with each year including and between the beginning year and the ending year.

23. The system of claim 22, wherein each study icon displays information regarding the respective associated study.

24. The system of claim 23, wherein the information includes one or more of a date, a modality, an anatomical structure, a referring physician, and a thumbnail of the study.

25. The system of claim 24, wherein the processors are further operable to receive a user request to change the displayed information; and
change the displayed information based on the user request.

26. The system of claim 22, wherein for each year that is associated with at least one study, the year icon associated with the respective year displays a number-of-studies indicator.

27. The system of claim 26, wherein the number-of-studies indicator is a bar having a length, the length being based on a number of studies associated with the respective year.

28. The system of claim 27, wherein the bar lengths are dynamically generated based on the number of studies associated with each year.

29. The system of claim 22, wherein the processors are further operable to receive a user selection of a first year icon associated with a selected year; and
scroll the scrollable list of studies to display the study icons associated with the selected year at a top of the scrollable list.

30. The system of claim 22, wherein the processors are further operable to receive a user selection of a first study from the studies associated with the patient; and
modify, on the graphical user interface, an appearance of the year icon associated with the year associated with the first study.

31. The system of claim 30, wherein the processors are further operable to identify one or more related studies from the studies associated with the patient, each related study being associated with the first study.

32. The system of claim 31, wherein the processors are further operable to modify, on the graphical user interface, the appearance of the year icon associated with each year associated with a related study, respectively.

33. One or more computer-readable non-transitory storage media embodying software that is operable when executed to:
receive a request to view two or more studies associated with a patient;
display, on a graphical user interface, a list of studies associated with the patient, the list including a study icon associated with each study;
receive a user selection of a first study from the studies associated with the patient;
modify, on the graphical user interface, an appearance of a first study icon associated with the first study;
display, on the graphical user interface, at least one image associated with the first study;
identify, without additional user input, one or more related studies from the studies associated with the patient, each related study being associated with the first study; and
modify, on the graphical user interface, an appearance of at least one study icon associated with one of the one or more related studies.

34. The storage media of claim 33, wherein each study icon displays information regarding the respective associated study.

35. The storage media of claim 34, wherein the displayed information includes one or more of a date, a modality, an anatomical structure, a referring physician, and a thumbnail of the study.

36. The storage media of claim 35, wherein the software is further operable to receive a user request to change the displayed information; and
change the displayed information based on the user request.

37. The storage media of claim 33, wherein identifying the one or more related studies from the studies associated with the patient is based at least in part on at least one of a procedure code associated with each study, an anatomical structure associated with each study, and a machine learning model.
